(19) United States
(12) Patent Application Publication
(10) Pub. No.: US 2023/0111706 A1
(43) Pub. Date: Apr. 13, 2023

(54) MEST AS BIOMARKER FOR CANCER DIAGNOSIS AND PROGNOSIS AND METHOD FOR USING THEREOF

(71) Applicant: KOREA INSTITUTE OF OCEAN AND SCIENCE & TECHNOLOGY, Gyeonggi-do (KR)

(72) Inventors: SUNG GYUN KANG, Gyeonggi-do (KR); HYUN SOOK LEE, Gyeonggi-do (KR); JUNG-HYUN LEE, Gyeonggi-do (KR); SANG-JIN KIM, Gyeonggi-do (KR); KAE KYONG KWON, Gyeonggi-do (KR); HYUNG-SOON YIM, Seoul (KR); YUN JAE KIM, Gyeonggi-do (KR); YOUNG OK HWANG, Gyeonggi-do (KR); WOOK JIN, Incheon-Si (KR); YONG KYUN CHO, Gyeonggi-Do (KR)

(21) Appl. No.: 18/078,391

(22) Filed: Dec. 9, 2022

Related U.S. Application Data
(62) Division of application No. 16/558,455, filed on Sep. 3, 2019.
Application No. 16/558,455 is a continuation-in-part of application No. 14/001,655, filed on Sep. 10, 2013, now abandoned.

(30) Foreign Application Priority Data
Feb. 25, 2011 (KR) 10-2011-0016983

(51) Int. Cl. C12Q 1/6886; G16H 50/30

(52) U.S. Cl. CPC C12Q 1/6886; G16H 50/30

(57) ABSTRACT

A method for treating cancer in a human subject includes initiating a cancer therapy on the human subject, preparing a biological sample from the human subject; (i) mixing the biological sample with an antibody or aptamer that specifically binds to an MEST protein, or (ii) obtaining mRNA of a nucleotide sequence encoding the MEST protein from the biological sample, and synthesizing and amplifying cDNA from the mRNA, detecting the MEST protein bound to the antibody or aptamer, or an expression of the cDNA, determining if the detected MEST or a level of the expression is higher than that in a normal human subject, determining responsiveness of the cancer to the cancer therapy by the detected MEST or the level of the expression, and continuing the cancer therapy to treat the cancer if it is determined that there is the responsiveness of the cancer to the cancer therapy.

### CROSS REFERENCE TO RELATED APPLICATIONS AND CLAIM OF PRIORITY

This application is a Divisional application of application Ser. No. 16/558,455, filed on Sep. 3, 2019, which is a Continuation-In-Part application of U.S. application Ser. No. 14/001,655, filed on Sep. 10, 2013, which is a National Phase application under 35 U.S.C. § 371 of International Application No. PCT/KR2012/001471, filed Feb. 27, 2012, which claims priority to Korean Patent Application No. 10-2011-0016983 filed Feb. 25, 2011, entire contents of which are incorporated herein by reference.

### BACKGROUND

### 1. Technical Field

The present invention relates to biomarkers for cancer diagnosis and prognosis and method for using biomarkers.

### 2. Background Art

Studies on cancer diagnosis together with studies on cancer treatment are receiving a great deal of attention in the fields of molecular biology and medicine. Although there were numerous studies on cancer diagnosis, a method capable of diagnosing cancer with certainty without any surgical operation has not yet been developed. With the development of molecular biology, studies on cancer diagnosis have been particularly focused on genetic defects and biomarkers (Dong et al., Science, 268:884 (1995)). For example, there have been cancer diagnosis studies on the transformation of ras oncogene, the amplification of HER-2/neu, the deletion and mutation of p53, the deletion of DCC and the mutation of BRCA1.

Malignant tumor (cancer) is the second leading cause of death following heart disease in USA (see Boring et al., CA Cancer J. Clin. 43:7 (1993)). Cancer is characterized by an increase in the number of abnormal or neoplastic cells derived from a normal tissue that proliferate to form tumor masses and that causes malignant cells that invade adjacent tissues and eventually metastasize via the blood or lymphatic system to local lymph nodes and distal portions. Cancerous cells grow even under conditions where normal cells do not grow. Cancer appears in highly diverse forms characterized by different degrees of invasiveness and metastatic potential.

In an attempt to find cellular targets effective for the diagnosis and treatment of cancer, researchers made efforts to find transmembrane polypeptides or membrane-binding polypeptides that are expressed more abundantly on the surface of one or more specific types of cancer cells than in one or more normal non-cancerous cells. Typically, such membrane-binding polypeptides are expressed more abundantly on the surface of cancer cells than on the surface of non-cancerous cells. By identifying antigenic polypeptides on the surface of such tumor-associated cells, cancer cells could be specifically targeted and killed using antibody-based therapies. Herein, the antibody-based therapies were demonstrated to be very effective for the treatment of specific cancers. For example, HERCEPTIN® and RITUXAN® (Genentech, Inc., South San. Calif., USA) are antibodies that have been successfully to treat breast cancer and non-Hodgkin's lymphoma. More specifically, HERCEPTIN® is a recombinant DNA-derived humanized monoclonal antibody that binds specifically to the extracellular domain of the human epidermal growth factor (HER2) proto-oncogene. Over-expression of the HER2 protein is observed in 25-30% of primary breast cancer. RITUXAN® is a genetically engineered chimeric murine/human monoclonal antibody to CD20 antigen that is found on the surface of normal B lymphocytes and malignant B lymphocytes. These two antibodies are all prepared in Chinese hamster ovary (CHO) cells by a recombinant method.

Meanwhile, genetic defects make it impossible to accurately diagnose cancer patients, frequently show positive results even in normal persons, and mostly require direct sampling of suspected tissue.

As patents related to cancer diagnosis, U.S. Pat. No. 5,942,385 disclosed a method for diagnosing metastatic cancer using VEGF (vascular endothelial growth factor) as a marker. U.S. Pat. No. 6,171,796 disclosed a method for diagnosing metastatic prostate cancer using transglutaminase or the like. U.S. Pat. No. 6,190,857 disclosed a method for diagnosing prostate cancer using interleukin-8 or interleukin-10 as a biomarker.

Accordingly, there is a need for the development of novel biomarkers capable of diagnosing cancer in a rapid and accurate manner.

Throughout the specification, a number of publications and patent documents are referred to and cited. The disclosure of the cited publications and patent documents is incorporated to herein by reference in its entirety to more clearly describe the state of the related art and the present disclosure.

### SUMMARY

The present inventors have made extensive efforts to discover novel biomarkers capable of diagnosing cancer in a rapid and accurate manner. As a result, the present inventors have found that the discovered biomarker can diagnose cancers and make cancer prognosis, thereby completing the present invention.

Therefore, it is an aspect of the present invention to provide a kit for cancer diagnosis and prognosis.

Another aspect of the present invention is to provide a method for detecting biomarkers required for cancer diagnosis and prognosis.

Other objects and advantages of the present invention will be more clearly understood from the following detailed description of the invention, the claims and the accompanying drawings.

In one aspect, the present invention provides a kit for cancer diagnosis and prognosis, the kit comprising: an antibody or aptamer that specifically binds to MEST protein; a nucleotide sequence that encodes MEST protein; and a sequence complementary to the nucleotide sequence or a fragment of the nucleotide sequence.

In another aspect, the present invention provides a method for detecting biomarkers required for cancer diagnosis or prognosis, the method comprising: detecting the expression of MEST protein or nucleotide sequence encoding MEST protein in a human biological sample.

In another aspect, a method for treating cancer in a human subject includes initiating a cancer therapy on the human subject, preparing a biological sample from the human subject; (i) mixing the biological sample with an antibody or aptamer that specifically binds to an MEST protein, or (ii) obtaining mRNA of a nucleotide sequence encoding the MEST protein from the biological sample, and synthesizing and amplifying cDNA from the mRNA, detecting the MEST protein bound to the antibody or aptamer, or an expression of the cDNA, determining if the detected MEST or a level of the expression is higher than that in a normal human subject, determining responsiveness of the cancer to the cancer therapy by the detected MEST or the level of the expression, and continuing the cancer therapy to treat the cancer if it is determined that there is the responsiveness of the cancer to the cancer therapy. The amino acid sequence of the MEST protein is selected from the group consisting of SEQ ID NO: 27 and SEQ ID NO: 28, and a nucleotide sequence of the mRNA is SEQ ID NO: 24 or SEQ ID NO: 25.

### BRIEF DESCRIPTION OF THE DRAWINGS

FIG. 1 showed that the expression of MEST in Mouse and Human Breast Cancer Cell Lines was examined by RT-PCT and Immunoblotting. HMLE—Normal Human Mammary Epithelial Cell; Hs578T—Human Breast Adenocarcinoma Cell; MDA-MB-231—Human Breast Adenocarcinoma Cell; MDA-MB-468—Human Breast Adenocarcinoma Cell; BT-474—Human Breast Ductal Carcinoma Cell; SKBR3—Human Breast Adenocarcinoma Cell; and ZR75-1—Human Breast Ductal Carcinoma Cell.

FIG. 2 showed that MEST was over-expressed in Human Breast Carcinoma. The relative expression levels of MEST in individual 17 human normal or invasive breast carcinoma samples were assessed by TaqMan real-time quantitative PCR. Expression was compared with that of healthy tissue. The endogenous 18S rRNA level was measured as the internal control. Black bar—healthy tissues; and white bar—patients' samples. Error bars represent mean±standard deviation of triplicate experiments.

FIG. 3 showed that Immunohistochemical analysis (IHC) for the level of MEST protein was performed in Normal Human Breast Cell and Infiltrating Duct Carcinoma (400× magnification). The scale bar represents 200 μm.

FIG. 4A showed that MEST induced the Epithelial-Mesenchymal Transition (EMT). The expression level of the MEST, fibronectin, a-catenin, ß-catenin, Twist-1, E-cadherin (Ecad) and N-cadherin (Ncad) was analyzed in HMLE (indicates ‘C’) and MEST-overexpressing HMLE (indicates ‘MEST’). HMLE means Normal Human Mammary Epithelial Cell. ß-actin was used to normalize the variability in template loading.

FIG. 4B showed that the relative expression level of occludin, claudin and CAR was determined by RT-PCR in HMLE (indicates ‘C’) and MEST-overexpressing HMLE (indicates ‘MEST’). ß-actin was used to normalize the variability in template loading.

FIG. 4C showed that the relative expression level of vimentin (Vim), E-cadherin (E-Cad), N-cadherin (N-Cad) and fibronectin (FN1) was determined by quantitative RT-PCR in HMLE (indicates ‘C’) and MEST-overexpressing HMLE (indicates ‘MEST’). 18S rRNA was used to normalize the variability in template loading.

FIG. 4D showed that the relative expression level of transcription factors like Snail, Slug, Twist-1 and Twist-2 was determined by quantitative RT-PCR in HMLE (indicates ‘C’) and MEST-overexpressing HMLE (indicates ‘MEST’). 18S rRNA was used to normalize the variability in template loading.

FIG. 5 showed that MEST localized in cytoplasm, not in mitochondria. HMLE and MEST-overexpressing HMLE (HMLE-MEST) were fixed with neutrally buffered 4% (w/v) paraformaldehyde, permeabilized with 0.2% Triton X-100 for 1 hour, and labeled with DAPI, MitoTracker (Mito.), V5, and subsequently rhodamin-conjugated secondary IgG. The cells were analyzed by confocal microscopy (LSM510, Zeiss).

FIG. 6 showed that MEST induced the Epithelial-Mesenchymal Transition (EMT). Immunofluorescence staining of V5, fibronectin, α-catenin, ß-catenin, E-cadherin, N-cadherin and Twist was performed in HMLE and MEST-overexpressing HMLE (HMLE-MEST). The red signal represents the staining of the corresponding protein, and the blue signal represents the nuclear DNA staining by DAPI.

FIG. 7A showed that the suppression of TrkC expression by stable MEST-siRNA reduced the cell proliferation. The protein level and RNA level of TrkC was examined by immunoblotting (Western) and RT-PCR in 4T1 cells which was stably expressing control siRNA (indicates ‘C’) or MEST-siRNA (indicates ‘siMEST’). The endogenous ß-actin and Gapdh mRNA levels were measured as the internal controls.

FIG. 7B showed the population doublings in wild-type 4T1 and 4T1 expressing MEST-siRNA. Each data point represents the mean of the number of cells in triplicate.

FIG. 8 showed the expression of MEST mRNA in normal human liver cell line and human liver carcinomas. The expression of MEST mRNA in human nonmetastatic or metastatic cell lines was examined by RT-PCR. The endogenous ß-actin mRNA level was measured as the internal control. Chang means normal human mammary eptihelial cells; SNU182, SNU354, SNU-368, SNU-387, SNU-449, and SNU-761 are human hepatocellular carcinoma cells derived from patients with liver cancer.

FIG. 9 showed that MEST was over-expressed in human liver carcinomas. The relative expression levels of MEST in individual 31 human normal or invasive liver carcinoma samples were assessed by TaqMan real-time PCR. Expression was compared with that of healthy tissue. The endogenous 18S rRNA level was measured as the internal control. Black bar—healthy tissues; and white bar—patients' samples.

### DETAILED DESCRIPTION

The present inventors have made extensive efforts to discover novel biomarkers capable of diagnosing cancer in a rapid and accurate manner, and as a result, have found that the disclosed molecular marker can easily diagnose cancer and make cancer prognosis. Particularly, the marker of the present invention has significantly improved accuracy and reliability.

MEST gene is located on human chromosome 7, the mRNA sequences for isoforms α and β are disclosed in NM 002402.2 (SEQ ID NO: 23), NM 177524.1 (SEQ ID NO: 24) and NM_177525.1 (SEQ ID NO: 25), respectively, and the protein sequences are disclosed in NP_002393.2 (SEQ ID NO: 26), NP_803490.1 (SEQ ID NO: 27) and NP_803491.1 (SEQ ID NO: 28), respectively.

As used herein, the term “biological sample” refers to any samples isolated from humans or mammals. Examples of the biological sample include, but are not limited to, cells, tissue, urine, sputum, blood, plasma or serum.

According to an embodiment of the present invention, the present invention is a cancer marker capable of diagnosing cancer from cells or tissue samples.

The molecular marker of the present invention can become an index of the development and progression of cancer and can be used to diagnose the development and progression of cancer.

According to an embodiment of the present invention, the molecular marker of the present invention is used to predict or diagnose any one or more cancers selected from the group consisting of breast cancer, liver cancer, bladder cancer, brain cancer, cervical cancer, colorectal cancer, esophageal cancer, gallbladder cancer, head and neck cancer, kidney cancer, lung cancer (small and/or non-small cell), melanoma, ovarian cancer, ovary (germ cell) cancer), prostate cancer, pancreatic cancer, penile cancer, skin cancer, soft-tissue sarcoma, squamous cell carcinomas, stomach cancer, testicular cancer, thyroid cancer and uterine cancer. More preferably, it is used to very accurately diagnose breast cancer, liver cancer, or both.

In addition, the present invention has characterized in accurately diagnosing metastatic cancer.

According to an embodiment of the present invention, the present invention is a marker for diagnosing metastatic cancer.

As used herein, the term “diagnosis” includes a determination of a subject's susceptibility to a disease or disorder, a determination as to whether a subject is presently affected by a disease or disorder, a prognosis of a subject affected by a disease or disorder (for example, identification of pre-metastatic or metastatic cancerous states, stages of cancer, or responsiveness of cancer to therapy), and therametrics (for example, monitoring a subject's condition to provide information as to the effect or efficacy of therapy).

As used herein, the term “prognosis” encompasses predictions about the likely course of disease, particularly with respect to likelihood of remission, relapse, tumor recurrence, metastasis, and death. Preferably, prognosis in the present invention refers to the likelihood for a disease in cancer patients to be perfectly cured.

According to an embodiment, the present invention can be performed by immunoassay, that is, an antigen-antibody reaction. In this case, the present invention is performed using an antibody or aptamer that specifically binds to the cancer marker of the present invention.

The antibody that is used in the present invention is a polyclonal or monoclonal antibody, preferably a monoclonal antibody. The antibody can be produced by methods generally known in the art, for example, a fusion method (Kohler and Milstein, European Journal of Immunology, 6:511-519(1976)), a recombinant DNA method (U.S. Pat. No. 481,656) or a phage antibody library method (Clackson et al, Nature, 352:624-628(1991) and Marks et al, J. Mol. Biol., 222:58, 1-597(1991)). General procedures for antibody production are described in detail in Harlow, E. and Lane, D., Using Antibodies: A Laboratory Manual, Cold Spring Harbor Press, New York, 1999; Zola, H., Monoclonal Antibodies: A Manual of Techniques, CRC Press, Inc., Boca Raton, Fla., 1984; and Coligan, CURRENT PROTOCOLS IN IMMUNOLOGY, Wiley/Greene, N Y, 1991, which are incorporated herein by reference.

For example, the production of hybridoma cells producing a monoclonal antibody is performed by fusing immortalized cells with antibody-producing lymphocytes, and the technology required for this procedure is well known to the skilled person in the art and can be easily performed. The polyclonal antibody can be obtained by injecting a protein antigen into a suitable animal, collecting anti-serum from the animal, and then isolating an antibody from the anti-serum by using known affinity technology.

When the method of the present invention is performed by using an antibody or an aptamer, the present invention can be used to diagnose cancer by an immunoassay.

This immunoassay can be performed according to various quantitative or qualitative immunoassay protocols known in the art. The immunoassay formats include, but not limited to, radioimmunoassay, radioimmunoprecipitation, immunoprecipitation, immunohistochemical staining, ELISA (enzyme-linked immunosorbent assay), capture-ELISA, inhibition or competition assay, sandwich assay, flow cytometry, immunofluorescent staining or immunoaffinity purification. The immunoassay or immunostaining methods are described in Enzyme Immunoassay, E. T. Maggio, ed., CRC Press, Boca Raton, Fla., 1980; Gaastra, W., Enzyme-linked immunosorbent assay (ELISA), in Methods in Molecular Biology, Vol. 1, Walker, J. M. ed., Humana Press, N J, 1984; and Ed Harlow and David Lane, Using Antibodies: A Laboratory Manual, Cold Spring Harbor Laboratory Press, 1999, which are incorporated herein by reference.

For example, when the method of the present invention is performed by radioimmunoprecipitation, an antibody labeled with radioactive isotopes (e.g., C₁₄, I₁₂₅, P₃₂ and S₃₅) can be used to detect the marker molecule of the present invention.

When the method of the present invention is performed by ELISA, a particular embodiment of the present invention comprises the steps of (i) coating the surface of a solid substrate with an unknown cell lysate to be analyzed; (ii) allowing a primary antibody to the marker to react with the cell lysates; (iii) allowing the material resulting from step (ii) to react with an enzyme-conjugated secondary antibody; and (iv) measuring the activity of the enzyme.

The solid substrate is preferably a hydrocarbon polymer (e.g., polystyrene or polypropylene), glass, a metal or gel, and most preferably a microtiter plate.

Examples of the enzyme conjugated to the secondary antibody include, but are not limited to, enzymes that catalyze color development reactions, fluorescence reactions, light-emitting reactions or IR reactions, such as alkaline phosphatase, ß-galactosidase, horse radish peroxidase, luciferase and cytochrome P450. When the enzyme conjugated to the secondary antibody is alkaline phosphatase, color development reaction substrates such as bromochloroindolyl phosphate (BCIP), nitro blue tetrazolium (NBT), naphthol-AS-B1-phosphate and ECF (enhanced chemifluorescence) may be used. When horse radish proxidase is used as the enzyme, substrates such as chloronaphthol, aminoethylcarbazole, diaminobenzidine, D-luciferin, lucigenin (bis-N-methylacridnium nitrate), resorufin benzyl ether, luminal, Amplex Red reagent (10-acetal-3,7-dihydroxyphenoxazine), HYR (p-phenylenediamine-HCl and pyrocatechol), TMB (tetramethylbenzidine), ABTS (2,2′-azine-di[3-ethylbenzthiazoline sulfonate]), o-phenylenediamine (OPD) and naphthol/pyronin, glucose oxidase, t-NBT (nitroblue tetrazolium) and m-PMS (phenazine methosulfate) may be used.

When the method of the present invention is performed by capture-ELISA, a particular embodiment of the present invention comprises the steps of: (i) coating the surface of a solid substrate with a capturing antibody to the marker of the present invention; (ii) reacting the capturing antibody with the sample; (iii) allowing the material resulting from step (ii) to react with a detecting antibody that has a signal-generating label bound thereto and responds specifically to MEST protein; and (iv) measuring a signal generated from the label.

The detecting antibody has a label that generates a detectable signal. Examples of the label include, but are not limited to, chemicals (e.g., biotin), enzymes (alkaline phosphatase, ß-galactosidase, horse radish peroxidase and cytochrome P450), radioactive substances (e.g., C14, I125, P32 and S35), fluorescent substances (e.g., fluorescein), light-emitting substances, chemiluminescent substances and FRET (fluorescence resonance energy transfer). Various labels and labeling methods are described in Ed. Harlow and David Lane, Using Antibodies: A Laboratory Manual, Cold Spring Harbor Laboratory Press, 1999.

In the ELISA method or capture-ELISA method, the measurement of activity of the enzyme or the measurement of the signal can be performed according to various methods known in the art. Detection of the signal enables the qualitative or quantitative analysis of the marker of the present invention. If biotin is used as the label, it can be easily detected with streptavidin, and if luciferase is used as the label, it can be easily detected with luciferin.

In an alternative embodiment of the present invention, an aptamer that specifically binds to the marker of the present invention may be used in place of the antibody. The aptamer is an oligonucleic acid or peptide, and general particulars of the aptamer are described in detail in Bock L C et al., Nature 355 (6360):5646 (1992); Hoppe-Seyler F, Butz K “Peptide aptamers: powerful new tools for molecular medicine”. J Mol Med. 78(8):42630(2000); Cohen B A, Colas P, Brent R. “An artificial cell-cycle inhibitor isolated from a combinatorial library”. Proc Natl Acad Sci USA. 95(24):142727 (1998).

Cancer can be diagnosed by analyzing the intensity of the signal resulting from the above-described immunoassay process. Specifically, when the marker protein of the present invention is highly expressed in a biological sample, and thus the signal is stronger in the biological sample than in a normal biological sample (e.g., normal stomach tissue, blood, plasma or serum), the biological sample is diagnosed as cancer.

The kit of the present invention may further comprise other components in addition to the above-described components. For example, when the kit of the present invention is applied to a PCR amplification process, it may optionally comprise reagents required for PCR amplification, for example, buffer, DNA polymerase (e.g., heat-stable DNA polymerase obtained from Thermus aquaticus (Taq), Thermus thermophilus (Tth), Thermus filiformis, Thermus flavus, Thermococcus literalis or Pyrococcus furiosus (Pfu)), DNA polymerase cofactor and dNTPs. The kit of the present invention may be made of a plurality of separate packagings or compartments including the above reagent components.

In an embodiment of the present invention, the kit of the present invention may be a microarray.

In an embodiment of the present invention, the kit of the present invention may be a gene amplification kit.

When the kit of the present invention is a microarray, a probe is immobilized on the solid surface of the microarray. When the kit of the present invention is a gene amplification kit, it comprises a primer.

The probe or primer that is used in the diagnostic kit of the present invention has a sequence complementary to the nucleotide sequence of MEST. As used herein, the term “complementary” refers to a sequence having complementarity to the extent that the sequence hybridizes or anneals specifically with the nucleotide sequence described above under certain hybridization or annealing conditions. In this regard, the term “complementary” has different meaning from the term “perfectly complementary”. The primer or probe of this invention may include one or more mismatch base sequences where it is able to specifically hybridize with the above-described nucleotide sequences.

The term “primer” used herein means a single-stranded oligonucleotide which is capable of acting as a point of initiation of template-directed DNA synthesis when placed under proper conditions (i.e., in the presence of four different nucleoside triphosphates and a thermostable enzyme) in an appropriate buffer and at a suitable temperature. The suitable length of primers will depend on many factors, including temperature, application and source of primer, generally, 15-30 nucleotides in length. In general, shorter primers need lower temperature to form stable hybridization duplexes to templates.

The sequences of primers are not required to have perfectly complementary sequence to templates. The sequences of primers may comprise some mismatches, so long as they can be hybridized with templates and serve as primers. Therefore, the primers of this invention are not required to have perfectly complementary sequence to the nucleotide sequence as described above; it is sufficient that they have complementarity to the extent that they anneals specifically to the nucleotide sequence of the gene for acting as a point of initiation of synthesis. The primer design may be conveniently performed with referring to the above-described nucleotide sequences. For instance, the primer design may be carried out using computer programs for primer design (e.g., PRIMER 3 program).

The term “probe” used herein refers to a linear oligomer of natural or modified monomers or linkages, including deoxyribonucleotides, ribonucleotides and the like, which is capable of specifically hybridizing with a target nucleotide sequence, whether occurring naturally or produced synthetically. The probe used in the present invention is preferably single-stranded and is an oligodeoxyribonucleotide.

To prepare primers or probes, the nucleotide sequence of the marker of the present invention may be found in the GenBank using the above-described accession numbers of MEST, and primers or probes may be designed by referencing the nucleotide sequence.

In the microarray of the present invention, the above probes serve as a hybridizable array element and are immobilized on a substrate. A preferable substrate includes suitable solid or semi-solid supporters, such as membrane, filter, chip, slide, wafer, fiber, magnetic or nonmagnetic bead, gel, tubing, plate, macromolecule, microparticle and capillary tube. The hybridizable array elements are arranged and immobilized on the substrate. Such immobilization occurs through chemical binding or covalent binding such as UV. In an embodiment of this invention, the hybridizable array elements are bound to a glass surface modified to contain epoxy compound or aldehyde group or to a polylysin-coated surface using UV. Further, the hybridizable array elements are bound to a substrate through linkers (e.g., ethylene glycol oligomer and diamine).

Meanwhile, a sample DNA that is applied to the microarray of the present invention may be labeled and is hybridized with the array element on the microarray. Various hybridization conditions are applicable, and for the detection and analysis of the extent of hybridization, various methods are available depending on the labels used.

The inventive kit for diagnosing cancer may be carried out in accordance with hybridization. In this case, probes having a sequence complementary to the nucleotide sequence of the marker of the present invention are used.

Using probes hybridizable with the nucleotide sequence of the marker of the present invention, cancer may be diagnosed by a hybridization-based assay.

The label of the probe may generate a signal to detect hybridization and may be linked to an oligonucleotide. Suitable labels include, but are not limited to, fluorophores (e.g., fluorescein, phycoerythrin, rhodamine, lissamine, Cy3 and Cy5 (Pharmacia)), chromophores, chemiluminescents, magnetic particles, radioisotopes (e.g., P³² and S³⁵), mass labels, electron dense particles, enzymes (e.g., alkaline phosphatase or horseradish peroxidase), cofactors, substrates for enzymes, heavy metals (e.g., gold), and haptens having specific binding partners, e.g., an antibody, streptavidin, biotin, digoxigenin and chelating group. Labeling is performed according to various methods known in the art, such as nick translation, random priming (Multiprime DNA labeling systems booklet, “Amersham” (1989)) and kination (Maxam & Gilbert, Methods in Enzymology, 65: 499 (1986)). The labels generate a signal detectable by fluorescence, radioactivity, measurement of color development, mass measurement, X-ray diffraction or absorption, magnetic force, enzymatic activity, mass analysis, binding affinity, high frequency hybridization or nanocrystal.

The nucleic acid sample to be analyzed may be prepared using mRNA from various biosamples, preferably mRNA from stomach tissue cells. Instead of probes, cDNA of interest may be labeled for hybridization-based analysis.

When probes are used, the probes are hybridized with cDNA molecules. Suitable hybridization conditions may be routinely determined by optimization procedures. To establish a protocol for use of laboratory, these procedures may be carried out by various methods known to those ordinarily skilled in the art. Conditions such as temperature, concentration of components, hybridization and washing times, buffer components, and their pH and ionic strength may be varied depending on various factors, including the length and GC content of probes and target nucleotide sequence. The detailed conditions for hybridization can be found in Joseph Sambrook, et al., Molecular Coning, A Laboratory Manual, Cold Spring Harbor Laboratory Press, Cold Spring Harbor, N.Y. (2001); and M. L. M. Anderson, Nucleic Acid Hybridization, Springer-Verlag New York Inc. N.Y. (1999). For example, the high stringent condition includes hybridization in 0.5 M NaHPO₄, 7% SDS (sodium dodecyl sulfate) and 1 mM EDTA at 65° C. and washing with 0.1×SSC (standard saline citrate)/0.1% SDS at 68° C. Also, the high stringent condition includes washing with 6×SSC/0.05% sodium pyrophosphate at 48° C. The low stringent condition includes e.g., washing with 0.2×SSC/0.1% SDS at 42° C.

Following hybridization reactions, a hybridization signal indicative of the occurrence of hybridization is then measured. The hybridization signal may be analyzed by a variety of methods depending on labels. For example, where probes are labeled with enzymes, the occurrence of hybridization may be detected by reacting substrates for enzymes with hybridization resultants. The enzyme/substrate pair useful in this invention includes, but is not limited to, a pair of peroxidase (e.g., horseradish peroxidase) and chloronaphthol, aminoethylcarbazol, diaminobenzidine, D-luciferin, lucigenin (bis-N-methylacridinium nitrate), resorufin benzyl ether, luminol, Amplex Red reagent (10-acetyl-3,7-dihydroxyphenoxazine), HYR (p-phenylenediamine-HCl and pyrocatechol), TMB (3,3,5,5-tetramethylbenzidine), ABTS (2,2-Azine-di[3-ethylbenzthiazoline sulfonate]), o-phenylenediamine (OPD) and naphthol/pyronine; a pair of alkaline phosphatase and bromochloroindolylphosphate (BCIP), nitro blue tetrazolium (NBT), naphthol-AS-B1-phosphate and ECF substrate; and a pair of glucose oxidase and t-NBT (nitroblue tetrazolium) or m-PMS (phenzaine methosulfate). Where probes are labeled with gold particles, the occurrence of hybridization may be detected by silver staining method using silver nitrate. Thus, where the inventive method for detecting the cancer marker is carried out by hybridization, it comprises the steps of (i) hybridizing a nucleic acid sample to a probe having a nucleotide sequence complementary to the nucleotide sequence of the marker of the present invention; and (ii) detecting the occurrence of the hybridization reaction. The intensity of the signal from hybridization is indicative of cancer. When the hybridization signal to the nucleotide sequence of the marker of the present invention from a sample to be diagnosed is measured to be stronger than normal samples (e.g., normal stomach tissue cells), the sample can be determined to have cancer.

The term “amplification” as used herein refers to reactions for amplifying nucleic acid molecules. A variety of amplification reactions have been reported in the art, and examples thereof include, but are not limited to, polymerase chain reaction (hereinafter referred to as PCR) (U.S. Pat. Nos. 4,683,195, 4,683,202, and 4,800,159), reverse transcription-polymerase chain reaction (hereinafter referred to as RT-PCR) (Sambrook, J. et al., Molecular Cloning. A Laboratory Manual, 3rd ed. Cold Spring Harbor Press (2001)), the methods of Miller, H. I. (WO 89/06700) and Davey, C. et al. (EP 329,822), ligase chain reaction (LCR), Gap-LCR (WO 90/01069), repair chain reaction (EP 439,182), transcription-mediated amplification (TMA; WO 88/10315), self sustained sequence replication (WO 90/06995), selective amplification of target polynucleotide sequences (U.S. Pat. No. 6,410,276), consensus sequence primed polymerase chain reaction (CP-PCR; U.S. Pat. No. 4,437,975), arbitrarily primed polymerase chain reaction (AP-PCR; U.S. Pat. Nos. 5,413,909 and 5,861,245), nucleic acid sequence based amplification (NASBA; U.S. Pat. Nos. 5,130,238, 5,409,818, 5,554,517 and 6,063,603), strand displacement amplification (21, 22) and loop-mediated isothermal amplification (LAMP) (23). Other amplification methods that may be used are described in U.S. Pat. Nos. 5,242,794, 5,494,810, 4,988,617 and in U.S. Ser. No. 09/854,317.

PCR is one of the most predominant processes for nucleic acid amplification and a number of its variations and applications have been developed. For example, to improve PCR specificity or sensitivity, touchdown PCR, hot start PCR, nested PCR and booster PCR have been developed by modifying traditional PCR procedures. In addition, real-time PCR, differential display PCR (DD-PCR), rapid amplification of cDNA ends (RACE), multiplex PCR, inverse polymerase chain reaction (IPCR), vectorette PCR and thermal asymmetric interlaced PCR (TAIL-PCR) have been developed for certain applications. The details of PCR can be found in McPherson, M. J., and Moller, S. G. PCR BIOS Scientific Publishers, Springer-Verlag New York Berlin Heidelberg, N.Y. (2000), the teachings of which are incorporated herein by reference in its entity.

Where the diagnostic kit of the present invention is used as primers, a gene amplification reaction is performed to examine the expression level of the nucleotide sequence of the inventive marker. Because the present invention is intended to analyze the expression level of the nucleotide sequence of the inventive marker, the mRNA level of the nucleotide sequence of the inventive marker to in a sample (e.g., stomach tissue, blood, plasma, serum or urine) is examined to determine the expression level of the nucleotide sequence of the inventive marker.

Thus, in the present invention, a gene amplification reaction is carried out using mRNA in a sample as a template and primers that bind to mRNA or cDNA.

To obtain mRNA, total RNA is isolated from a sample. The isolation of total RNA may be performed by conventional methods known in the art (see Sambrook, J. et al., Molecular Cloning. A Laboratory Manual, 3rd ed. Cold Spring Harbor Press (2001); Tesniere, C. et al., Plant Mol. Biol. Rep., 9: 242 (1991); Ausubel, F. M. et al., Current Protocols in Molecular Biology, John Willey & Sons (1987); and Chomczynski, P. et al., Anal. Biochem. 162: 156 (1987)). For example, total RNA in cells may be isolated using Trizol. Then, cDNA is synthesized from the isolated mRNA and then amplified. Because total RNA used in the present invention is isolated from a human sample, the ends of mRNA have poly-A tails, and cDNA can be easily synthesized using dT primers and reverse transcriptase (see PNAS USA, 85: 8998 (1988); Libert F, et al., Science, 244: 569 (1989); and Sambrook, J. et al., Molecular Cloning. A Laboratory Manual, 3rd ed. Cold Spring Harbor Press (2001)). The synthesized cDNA is then amplified by a gene amplification reaction.

The primers that are used in the present invention are hybridized or annealed to portions of the template to form a double-stranded structure. Nucleic acid hybridization conditions suitable for forming this double stranded structure are described in Joseph Sambrook, et al. Molecular Cloning, A Laboratory Manual, Cold Spring Harbor Laboratory Press, Cold Spring Harbor, N.Y. (2001) and Haymes, B. D., et al., Nucleic Acid Hybridization, A Practical Approach, IRL Press, Washington, D.C. (1985).

A variety of DNA polymerases may be used for amplification in the present invention, and examples thereof “Klenow” fragment of E. coli DNA polymerase I, thermostable DNA polymerase and bacteriophage T7 DNA polymerase. Preferably, the polymerase is thermostable DNA polymerase obtainable from a variety of bacterial species, including Thermus aquaticus (Taq), Thermus thermophiles (Tth), Thermus filiformis, Thermus flavus, Thermococcus literatis, and to Pyrococcus furiosus (Pfu).

When a polymerization reaction is performed, excess amounts of components required for the reaction are provided into the reactor. Herein, the term “excess amount” refers to an amount of a component such that the amplification reaction is not substantially limited by the concentration of that component. It is required to provide cofactors such as Mg 2⁺, and dATP, dCTP, dGTP and d HP to the reaction mixture so that a desired degree of amplification can be achieved. All the enzymes used in the amplification reaction may be active under the same reaction conditions. Indeed, buffers allow all the enzymes to approach the optimum reaction conditions. Therefore, the amplification process of the present invention can be performed in a single reaction without any change in conditions such as addition of reactants.

Annealing in the present invention is performed under stringent conditions that allow for specific binding between the target nucleotide sequence and the primers. Such stringent conditions for annealing will be sequence-dependent and vary depending on environmental parameters.

The amplified cDNA for the nucleotide sequence of the marker of the present invention is then analyzed to assess its expression level using suitable methods. For example, the amplified product is subjected to gel electrophoresis and the bands generated are observed and analyzed to determine the expression level of the nucleotide sequence of the marker of the present invention. When the expression level of the nucleotide sequence of the present marker in the sample is measured to be higher than normal samples (normal cells, blood, plasma or serum), the sample is diagnosed as cancer.

Thus, when the method for detecting the cancer marker of the present invention is carried out based on an amplification reaction, it comprises the steps of (i) performing an amplification using primers that are annealed to the nucleotide sequence of the marker of the present invention; and (ii) analyzing the product of the amplification reaction to determine the expression level of the nucleotide sequence of the marker.

The marker of the present invention is a biomolecule that is highly expressed in cancer. The high expression of the marker can be measured at the mRNA or protein level. As used herein, the term “high expression” means that the expression level of the nucleotide sequence of interest in a sample to be analyzed is higher than that in a normal sample. For example, the term means that the expression level of the nucleotide sequence of interest is determined to be higher when analyzed by a conventional analysis method known in the art, for example, RT-PCR or ELISA (see Sambrook, J. et al., Molecular Cloning. A Laboratory Manual, 3rd ed. Cold Spring Harbor Press (2001)). For example, when the expression level of the marker of the present invention in a sample is about 2-10 times higher than that in normal cells, as analyzed by the above analysis method, the sample is determined to be “high expression” and diagnosed as cancer.

The features and advantages of one or more embodiments of the present invention are summarized as follows:

(i) The present invention provides a kit for cancer diagnosis and prognosis.

(ii) MEST in the present invention is a marker having significantly improved accuracy and reliability. Particularly, the marker in the present invention has high accuracy and reliability for breast cancer or liver cancer.

(iii) The marker in the present invention has very high accuracy and reliability for metastatic cancer.

(iv) In addition, according to the present invention, the early cancer diagnosis and prognosis can be achieved by analyzing a biological sample (e.g., cells or tissue) because the expression of MEST specifically increases in the cells and tissues of cancer patients.

Hereinafter, the present invention will be described in further details with reference to examples. It is to be understood, however, that these examples are for illustrative purposes only and are not to be construed to limit the scope of the present invention.

Throughout the specification, “%” used to indicate the concentration of a particular substance is that, unless otherwise noted, solid/solid is (weight/weight) %, solid/liquid is (weight/volume) %, and liquid/liquid is (volume/volume) %.

### Example 1

Cell Culture, Antibodies and Reagents.

Mouse 4T1 cell line was cultured in high-glucose DMEM (Gibco, Grand Island, N.Y.) supplemented with 10% heat-inactivated fetal bovine serum (hereinafter “FBS”). Immortalized human mammary epithelial cells (hereinafter “HMLE”) was cultured in DMEM/F12 supplemented with insulin (10 μg/ml), human EGF (epidermal growth factor, 10 ng/ml), hydrocortisone (0.5 μg/ml) and 10% heat-inactivated FBS in a 37° C., 5% CO₂ incubator.

Anti-rabbit HRP-link IgG (7074), anti-rabbit HRP-link IgG (7076), and anti-mouse IgG were purchased from Cell Signaling Technology, anti-E-cadherin (61181). anti-N-cadherin (610920), anti-CD24(555428), anti-Cd44(555478), anti-fibronectin (610077) were purchased from BD sciences, anti-ß-catenin (13-9700), anti-α-catenin (13-8400) from Zymed, anti-Twist1 (sc-6269) from Santa Cruz technology. Also, anti-V5 (R96125), Mito Tracker (M7512), goat serum (50062Z), ProLong Gold antifade reagent with DAPI (P36935) and ViraPower Lentiviral packing mix (K4975-00) from Invitrogen. Anti-ß-actin (A1978), anti-MEST (HPA005623) were purchased from Sigma-Aldrich. pFG12 lentivirus vector was purchased from ADDGEGE. Chang normal liver cell and human liver carcinoma such as SNU182, SNU354, SNU368, SNU387, SNU449 and SNU761 were cultured in high-glucose DMEM (Gibco, Grand Island, N.Y.) supplemented with 10% heat-inactivated FBS in a 37° C., 5% CO₂ incubator.

293T cells, mouse breast adenocarcinoma cell (NMuMG, 67NR and 4T1 cell line), human breast adenocarcinoma cell (Hs578T, MDA-MB-231, MDA-MB-468, BT-474, SKBR3 and ZR75-1), normal human liver cell (Chang liver cell), SNU-182, SNU-387 and SNU-449 were purchased from ATCC (American Type Culture Collection), SNU-354, SNU-368 and SNU-761 were purchased from KCLB (Korean Cell Line Bank).

### Example 2

Human Tumor Samples.

RNAs isolated from Human tissue samples of normal and invasive breast carcinoma derived from patients were generously provided from Gangnam Severance Hospital, and the tissues were purchased from Imgenex.

### Example 3

MEST siRNA Plasmid.

For each two siRNA-coding oligo of mouse MEST, BLAST search from mouse genome was applied and MEST siRNA oligo targeting 5′-GCCCTTGATTTCTTAGGCTTT-3′ (SEQ ID NO: 1) and 5′-CCACATCAGTACTCCATATTT-3′ (SEQ ID NO: 2) was designed and confirmed.

For hairpin-type single RNAi vectors, 5 [symbol] of 100 mM synthetic sense and antisense oligonucleotide

(SEQ ID NO: 3)
(5′-CTAGACCCCACATCAGTACTCCATATTTCTCGAG
AAATATGGAGTACTGATGTGGTTTTTGGAAAC-3′)
and
(SEQ ID NO: 4)
(5′-CTAGACCGCCCTTGATTTCTTAGGCTTT
TTCAAGAGAAAAGCCTAAGAAATCAAGGGCTTTTTGGAAAC-3′)

were mixed with 1 [symbol] of 1 M NaCl. Then, annealing at 95° C. for 2 min, cooling at 72° C., and then slowly cooling at room temperature was performed.

Mouse MEST-siRNA inserts were sub-cloning to XbaI/XhoI loci of pFG12 lentivirus vector (Inhibiting HIV-1 infection in human T cells by lentiviral-mediated delivery of small interfering RNA against CCR5. Qin X F et al. (Proc Natl Acad Sci USA. 2003 Jan. 7. 100(1):183-8. Pubmed)). Control siRNA was manufactured by using the sequence which was known for not coding mouse cDNA.

### Example 4

RT-PCR

All RNAs were purified by using QIAzol lysis reagent (Qiagen, Inc., Valencia, Calif.). Reverse transcription was performed with one-stop RT-PCR kit (Qiagen, Inc., Valencia, Calif.). Each PCR product was analyzed on the 1% agarose gel. Forward primer (5′-TCAGTGACAAACCGAGACCA-3′) (SEQ ID NO: 5) and reverse primer (5′-CATCAGTCGTGTGAGGATGG-3′) (SEQ ID NO: 6) were used for MEST RT-PCR

### Example 5

Immunoblotting.

All proteins were purified from mouse breast adenocarcinoma cell (NMuMG, 67NR and 4T1 cell), MEST-overexpressing cell line (hereinafter “HMLE-MEST”), MEST-knockdown cell (4T1-siMEST), Chang liver cell, SNU-182, SNU-387, SNU-449, SNU-354, SNU-368 and SNU-761 by using buffer (25 mM Hepes (pH 7.5), 150 mM NaCl, 1% Triton X-100, 10% glycerol, 5 mM EDTA, protease inhibitor mixtures (Complete, Roche, Gipf-Oberfrick, Switzerland)).

Purified proteins were separated on SDS/PAGE, transferred to PVDF (polyvinylidene difluoride) membrane, and then incubated with polyclonal or monoclonal 1^{st} antibodies (anti-MEST (HPA005623): Sigma-Aldrich). Then, the membrane was incubated with 2^{nd} HRP (horseradish peroxidase)-conjugated anti-rabbit and anti-mouse IgG. The target proteins were confirmed by chemiluminescent detection method according to the manufacturer's instructions (Pierce).

### Example 6

Viral Production and Infection of Target Cell.

Transfer vector plasmid pFG12-siLuc (empty) or pFG12-mouse siMEST were mixed with ViraPower Lentiviral Packing Mix and transfected to 293T cells by using calcium phosphate methods.

Supernatants were transfected for 72 hours, collected by 0.45 μm filters, centrifuged at 100,000×g by using SW28 Rotor, and suspended by 100 [symbol] of 0.1% bovine serum albumin (BSA) in phosphate-buffered saline (hereinafter “PBS”) buffer. Lentivirus stocks were stored at −80° C. freezer before use. For the cellular infection, mouse breast adenocarcinoma cells (4T1 cell lines) were cultured for 12 hours after inoculating at 6-well plates (1×10⁵ cells/well). Lentivirus was added to 2 ml of DMEM supplemented 8 μg/ [symbol] of polybrene and centrifuged at 1,500 rpm for 30 minutes. After 24 hours from the infection, polybrene-DMEM was replaced with new DMEM.

### Example 7

Quantitative RT-PCR

Forward primer: TGCCCAGAAAATGAAAAAGG (SEQ ID NO: 7), reverse primer: GTGTATGTGGCAATGCGTTC (SEQ ID NO: 8) was used for E-cadherin; forward primer: ACAGTGGCCACCTACAAAGG (SEQ ID NO: 9), reverse primer: CCGAGATGGGGTTGATAATG (SEQ ID NO: 10) for N-cadherin; forward primer: CAGTGGGAGACCTCGAGAAG (SEQ ID NO: 11), reverse primer: TCCCTCGGAACATCAGAAAC (SEQ ID NO: 12) for fibronectin; forward primer: GAGAACTTTGCCGTTGAAGC (SEQ ID NO: 13), reverse primer: GCTTCCTGTAGGTGGCAATC (SEQ ID NO: 14) for vimentin. Also, for the activity of EMT-inducing transcription factors, forward primer: CCTCCCTGTCAGATGAGGAC (SEQ ID NO: 15), reverse primer: CCAGGCTGAGGTATTCCTTG (SEQ ID NO: 16) were used for Snail; forward primer: GGGGAGAAGCCTTTTTCTTG (SEQ ID NO: 17), reverse primer: TCCTCATGTTTGTGCAGGAG (SEQ ID NO: 18) for Slug; forward primer: CGACGAGCTGGACTCCAAG (SEQ ID NO: 19), reverse primer: CCTCCATCCTCCAGACCGA (SEQ ID NO: 20) for Twist-1; forward primer: CAGAGCGACGAGATGGACAA (SEQ ID NO: 21), reverse primer: CACACGGAGAAGGCGTAGC (SEQ ID NO: 22) for Twist-2.

Total RNAs were purified by using RNeasy mini-kit (Qiagen), and cDNA was produced by using hexa-nucleotide Mix (Roche). Then, cDNA was used for PCR by using SYBR-green Master PCR mix and TaqMan Master PCR Mix (Applied Biosystems). PCR data collection was used by 7900HT Fast Real-Time PCR system (Applied Biosystems). 18S rRNA was used as the endogenous control in all quantification. Relative Quantification of each target gene was indicated as 2^{ΔΔCT} (CT—cycle threshold). MEST (Hs00853380_g1) and 18S (Hs03003631_g1) probes for quantitative TaqMan RT-RCR were purchased from Applied Biosystems.

### Example 8

Immunofluorescence.

After 24 hours from seeding 2.5×10⁴ cells of HMLE and HMLE-MEST on 4-well Lab-TekII chamber slides, cells were washed twice with PBS. Then, cells were treated with 2% of paraformaldehyde and 0.1% of Triton X-100 in PBS, fixed for 30 minutes, and then washed three times with PBS. Cells were treated with blocking solution (10% goat serum in PBS) and then incubated. After blocking, cells were incubated with 1^{st} antibody for 2 hours, washed three times with PBS including 0.1% Tween-20, incubated with 2^{nd} antibody and DAPI for 2 hours, and then mounted with Slowfade Light Antifade Kit (Invitrogen). All samples were measured by immunofluorescent microscopy in the same conditions.

### Example 9

Immunohistochemistry.

After tissue microarray slides (IMX-364) were deparaffinized and rehydrated, heat-induced epitope retrieval was performed with 0.01 mol/L of citric acid buffer (pH 6.0). Endogenous peroxidase activity was treated with 3% hydrogen peroxide for 10 minutes. Non-specific binding was used with 5% goat serum for 1 hour. After the slides were incubated with MEST antibody for 12 hours at 4° C., the images were measured by using LSAB2 system (DakoCytomation).

### Example 10

Expression of MEST Gene in Human Breast Adenocarcinoma Cells.

It was reported that MEST gene that is a newly identified imprinted gene has two isoforms that are made by spliced variant isoform mRNA. It was known that isoform 1 (long isoform) is expressed in the brain, skeletal muscles, kidneys, human organs, adrenal, tongues, hearts, skin and placenta, and isoform 2 (short isoform) is free of nine residues at the N-terminal end. In addition, it was reported that isoform 2 is expressed in several non-placenta organs, but the correlation thereof with cancer has not yet been reported.

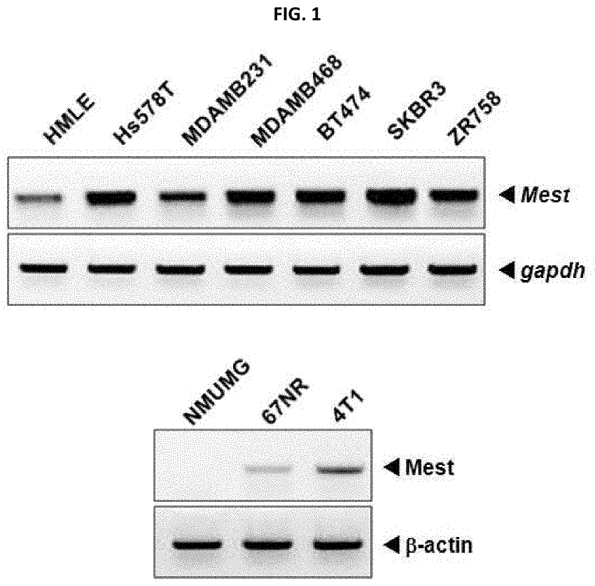

Thus, in order to examine the correlation between the expression of MEST gene and cancer, the expression of MEST gene in human breast cancer cell lines was examined. As seen in FIG. 1, the expression of MEST gene was higher in the breast cancer cell lines than in HMLE and was stronger in the breast cancer cell lines.

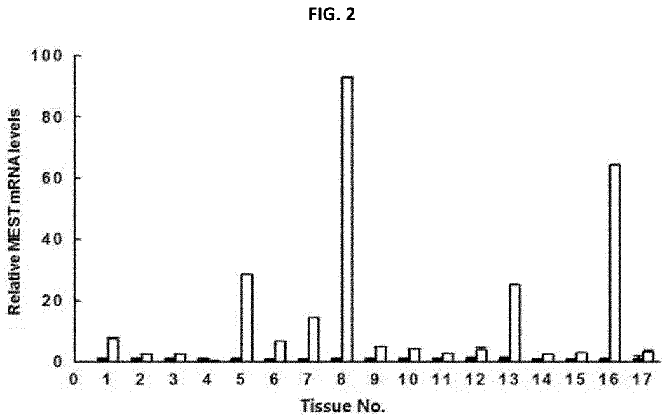

In addition, in order to examine whether the expression of MEST gene is related to any pathological phenotype in clinical breast cancer samples, RNAs were isolated from invasive human breast cancer tissues obtained form 17 patients. It was shown that the expression of the MEST gene in the normal tissues did greatly differ from that in cancer tissue of the patients. It was shown that the expression of the MEST gene greatly increased (2-96 times) in tissue samples of 16 patients among to 17 patients. Specifically, it was shown that MEST was over-expressed in 94% or more of the patients (FIG. 2). However, the expression of MESTb that is the MEST isoform was not detected in the breast cancer patient samples.

Based on such results, 57 tissue samples of breast cancer patients tissues were performed by immunohistochemistry with MEST antibody.

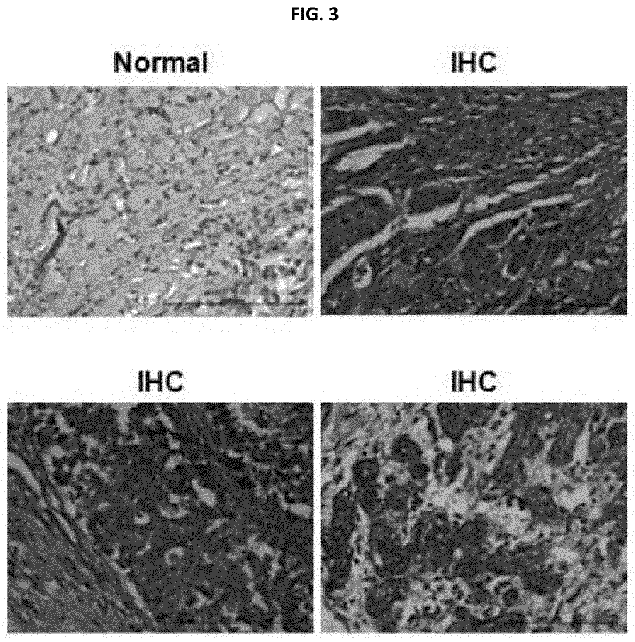

Normal human breast cells were very weakly stained with the MEST antibody, whereas infiltrating duct carcinoma (IDC) was strongly stained with the MEST antibody as follows: −/+: 5 samples; ++: 14 samples; and +++: 26 samples. Thus, it was shown that MEST was strongly expressed in most of the breast cancer tissues (FIG. 3).

### Example 11

Relationship Between MEST Expression and Cancer Stem Cell (CSC) and Induction of Epithelial-Mesenchymal Transition (EMT).

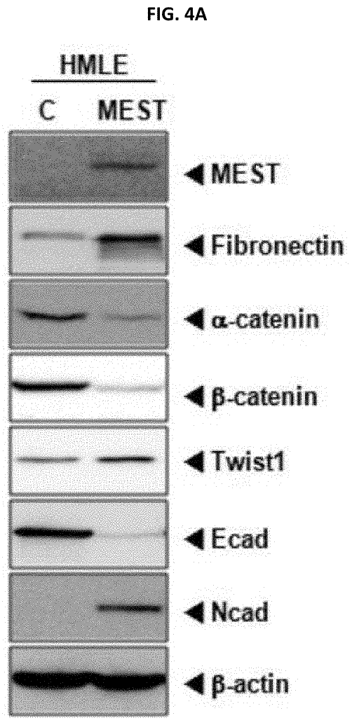

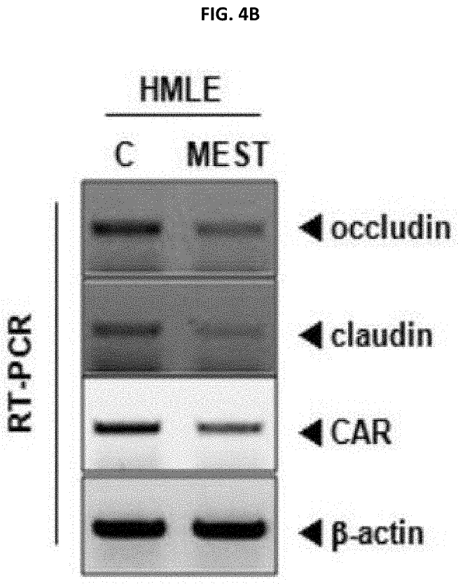

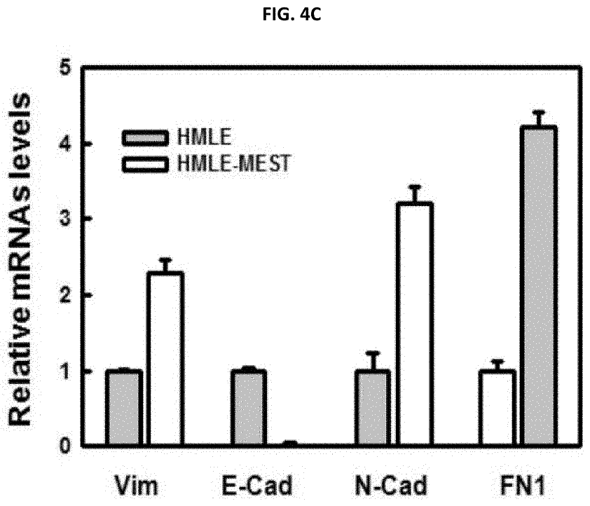

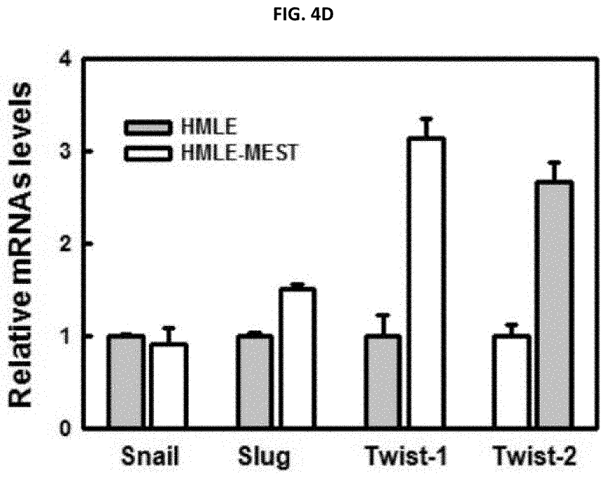

An important mechanism for the loss of E-cadherin mRNA is attributable to the inhibition of direct transcription by transcription factors such as E12, E47, SIP1, slug, Goosecoid, twist and so forth. Also, it was reported that these transcription factors are over-expressed in various human tumors and show a close relationship with tumor invasion or metastasis. Thus, the expression of the transcription factors that are involved in inducing EMT by the expression of MEST was analyzed by quantitative RT-PCR As a result, the expression of Snail in HMLE did not greatly differ from that in HMLE-MEST. However, it was shown that the expression of Slug was increased by about 1.8 times due to MEST, and the expression of Twist-1 and Twist-2 greatly increased (FIG. 4D).

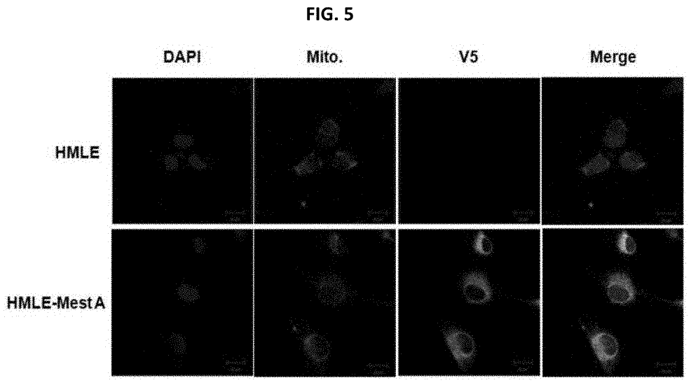

In this study, MEST was estimated to have putative mitochondria targeting peptides and a mitochondrial protein as determined using TargetP, iPsort and MitoProt programs. Thus, the intracellular position of MEST was examined. The results of staining MEST using Mito-Tracker were shown that MEST is not located in the mitochondria or the nucleus. Thus, it appears that MEST is located in the cytoplasm (FIG. 5).

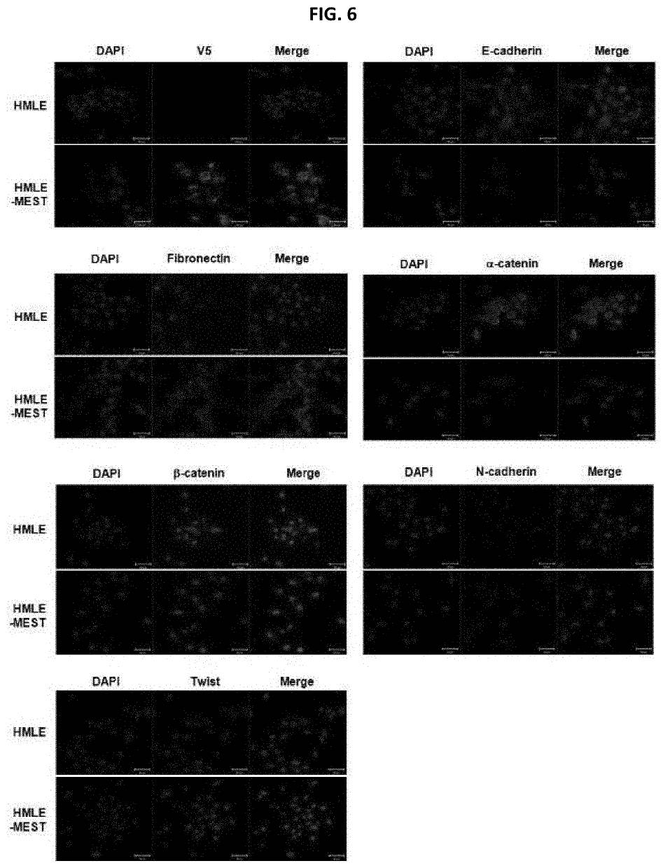

In order to examine whether EMT is induced by the expression of MEST, epithelial cell markers and mesenchymal markers were immunostained in HMLE and HMLE-MEST. As a result, it was shown that the expression of E-cadherin, α-catenin and ß-catenin (epithelial cell markers) was decreased according to the overexpression of MEST and that the expression of fibronectin and N-cadherin (mesenchymal markers) was increased (FIG. 6). In addition, it was shown that the transcription factor Twist-1 inducing EMT was more strongly immunostained according to the overexpression of MEST (FIG. 6).

### Example 12

Tumor Growth and Tumor Cell Viability Resulting from MEST Expression

In order to examine the functional role of MEST gene in breast tumor growth, a siRNA technique of knocking down the expression of the MEST gene was used in mouse breast adenocarcinoma (4T1 cell lines) showing a high expression level of the MEST gene. Specifically, siRNA for a region encoding the mouse MEST gene was designed, and as a control, siRNA for luciferase DNA that is not matched with the known mouse genes was designed.

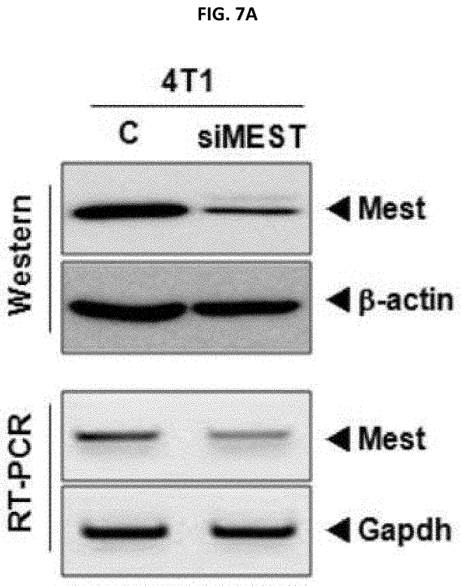

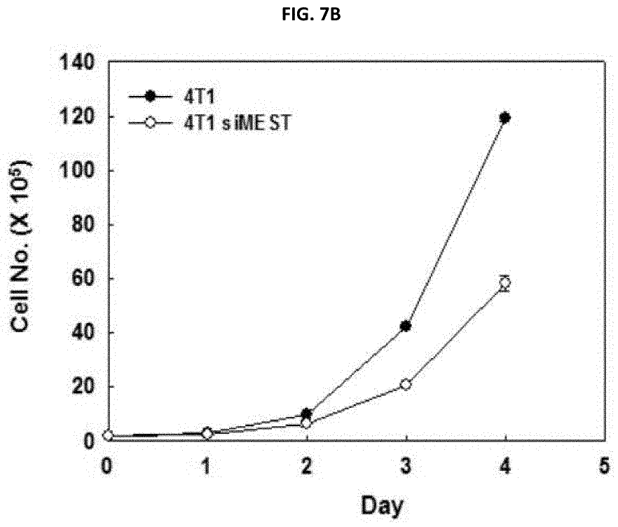

The expression of MEST mRNA and protein in the 4T1 cell lines transfected with siRNA was examined, and as a result, it was shown that the expression of MEST mRNA and protein in the 4T1 cell lines transfected with siRNA was significantly decreased as compared to that in the control group (FIG. 7A). Then, it was examined whether the knockdown of MEST expression influences cell growth. As a result, it was shown that the growth in 4T1 expressing MEST-siRNA was significantly decreased as compared to that in the control group (FIG. 7B). AKT (also known as Protein Kinase B) is a serine/threonine kinase and belongs to the cAMP-dependent protein-kinase A/protein kinase G/protein kinase C super-family. It was reported that the activation of AKT is induced in the process of signal transduction by growth factors or insulin and is involved in many intracellular processes such as cell growth and survival, glucose metabolism and transcription regulation.

In addition, it was reported that AKT is activated by phosphorylation at serine 308 and serine 473 by PI3K (Phosphatidylinositide 3-kinases), and according to this activation, AKT plays an important role in cell growth, survival and apoptosis and also induces continuous localization of many downstream pro-apoptosis protein targets. It is expected that the activation of AKT by over-expression of MEST will play an important role in breast cancer growth, survival and apoptosis.

### Example 13

Expression of MEST Gene in Human Liver Carcinoma Cell Lines.

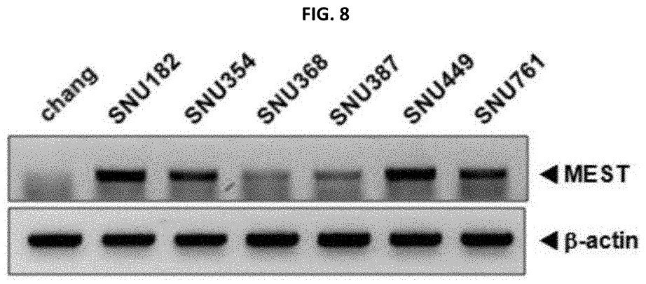

In order to examine the relationship between the expression of MEST gene and liver cancer, the expression of MEST gene in human liver carcinoma cell lines was analyzed. As a result, it was shown that the expression of MEST was higher in the liver carcinoma cells than in normal Chang liver cells used as a control group (FIG. 8).

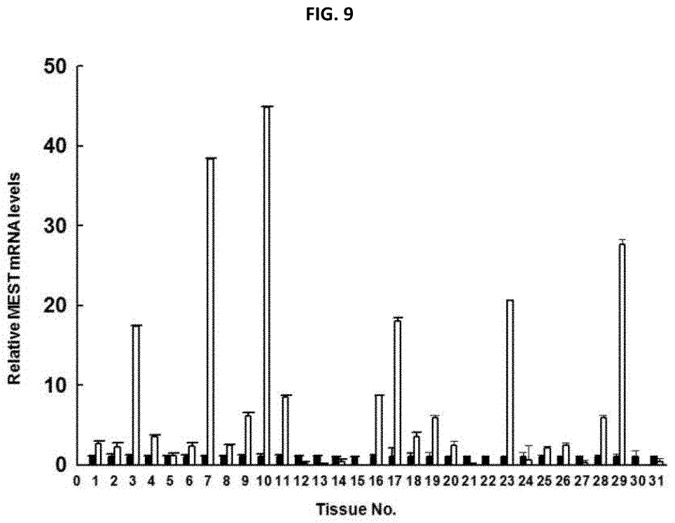

In addition, in order to examine whether the expression of MEST gene is related to any pathological phenotype in clinical liver tumor samples, RNAs were isolated from invasive human liver tissues obtained from 31 patients. As a result, it was shown that the expression of MEST in normal tissues did greatly differ from that in tumor tissues of the patients (FIG. 9). Further, it was shown that the expression of MEST was greatly increased (2-44 times) in tumor samples of 20 patients among 31 patients. Specifically, it was shown that MEST were over-expressed in 65% or more of the patients.

### REFERENCE TO AN ELECTRONIC SEQUENCE LISTING

A sequence listing electronically submitted on Dec. 9, 2022 as a XML file named 20221209_P27813YG-CIP-V_TU_SEQ.XML, created on Nov. 18, 2022 and having a size of 41,995 bytes, is incorporated herein by reference in its entirety.

What is claimed is:

1. A method for treating cancer in a human subject, the method comprising:
initiating a cancer therapy on the human subject;
preparing a biological sample from the human subject;
(i) mixing the biological sample with an antibody or aptamer that specifically binds to an MEST (mesoderm specific transcript homolog) protein, or (ii) obtaining mRNA of a nucleotide sequence encoding the MEST protein from the biological sample, and synthesizing and amplifying cDNA from the mRNA;
detecting the MEST (mesoderm specific transcript homolog) protein bound to the antibody or aptamer, or an expression of the cDNA;
determining if the detected MEST or a level of the expression is higher than that in a normal human subject;
determining responsiveness of the cancer to the cancer therapy by the detected MEST or the level of the expression; and
continuing the cancer therapy to treat the cancer if it is determined that there is the responsiveness of the cancer to the cancer therapy,
wherein an amino acid sequence of the MEST protein is selected from the group consisting of SEQ ID NO: 27 and SEQ ID NO: 28; and
a nucleotide sequence of the mRNA is SEQ ID NO: 24 or SEQ ID NO: 25.

2. The method of claim 1, wherein said cancer is selected from breast cancer, liver cancer, bladder cancer, brain cancer, cervical cancer, colorectal cancer, esophageal cancer, gallbladder cancer, head and neck cancer, kidney cancer, lung cancer (small and/or non-small cell), melanoma, ovarian cancer, ovary (germ cell) cancer, prostate cancer, pancreatic cancer, penile cancer, skin cancer, soft-tissue sarcoma, squamous cell carcinomas, stomach cancer, testicular cancer, thyroid cancer, uterine cancer, and a combination thereof.

3. The method of claim 1, wherein said cancer is metastatic cancer.

4. The method of claim 1, wherein the detection is performed by using a microarray having probes.

5. The method of claim 1, wherein the detection step comprises detecting the MEST protein.

6. The method of claim 4, wherein the cDNA or the probes are labeled with a label selected from the group consisting of a fluorophore, a chromophore, a chemiluminescent, magnetic particle, a radioisotope, a mass label, an electron dense particle, an enzyme, a cofactor, a substrate for enzyme, a heavy metal, and a hapten.

7. The method of claim 1, wherein the amino acid sequence of the MEST protein is SEQ ID NO: 27.

8. The method of claim 7, wherein a nucleotide sequence of the mRNA is SEQ ID NO: 24.

9. The method of claim 1, wherein the amino acid sequence of the MEST protein is SEQ ID NO: 28.

10. The method of claim 9, wherein a nucleotide sequence of the mRNA is SEQ ID NO: 25.


SEQUENCE LISTING (28 sequences)

SEQ ID NO: 1 - DNA, 21 bp, Mus musculus
  source 1..21: mol_type = genomic DNA
gcccttgatttcttaggcttt

SEQ ID NO: 2 - DNA, 21 bp, Mus musculus
  source 1..21: mol_type = genomic DNA
ccacatcagtactccatattt

SEQ ID NO: 3 - DNA, 66 bp, synthetic construct
  misc_feature 1..66: note = synthetic sense and antisense oligonucleotide for hairpin-type single RNAi vectors
  source 1..66: mol_type = other DNA
ctagaccccacatcagtactccatatttctcgagaaatatggagtactgatgtggtttttggaaac

SEQ ID NO: 4 - DNA, 69 bp, synthetic construct
  misc_feature 1..69: note = synthetic sense and antisense oligonucleotide for hairpin-type single RNAi vectors
  source 1..69: mol_type = other DNA
ctagaccgcccttgatttcttaggctttttcaagagaaaagcctaagaaatcaagggctttttggaaac

SEQ ID NO: 5 - DNA, 20 bp, synthetic construct
  misc_feature 1..20: note = forward primer for MEST RT-PCR
  source 1..20: mol_type = other DNA
tcagtgacaaaccgagacca

SEQ ID NO: 6 - DNA, 20 bp, synthetic construct
  misc_feature 1..20: note = reverse primer for MEST RT-PCR
  source 1..20: mol_type = other DNA
catcagtcgtgtgaggatgg

SEQ ID NO: 7 - DNA, 20 bp, synthetic construct
  misc_feature 1..20: note = forward primer for E-cadherin
  source 1..20: mol_type = other DNA
tgcccagaaaatgaaaaagg

SEQ ID NO: 8 - DNA, 20 bp, synthetic construct
  misc_feature 1..20: note = reverse primer for E-cadherin
  source 1..20: mol_type = other DNA
gtgtatgtggcaatgcgttc

SEQ ID NO: 9 - DNA, 20 bp, synthetic construct
  misc_feature 1..20: note = forward primer for N-cadherin
  source 1..20: mol_type = other DNA
acagtggccacctacaaagg

SEQ ID NO: 10 - DNA, 20 bp, synthetic construct
  misc_feature 1..20: note = reverse primer for N-cadherin
  source 1..20: mol_type = other DNA
ccgagatggggttgataatg

SEQ ID NO: 11 - DNA, 20 bp, synthetic construct
  misc_feature 1..20: note = forward primer for fibronectin
  source 1..20: mol_type = other DNA
cagtgggagacctcgagaag

SEQ ID NO: 12 - DNA, 20 bp, synthetic construct
  misc_feature 1..20: note = reverse primer for fibronectin
  source 1..20: mol_type = other DNA
tccctcggaacatcagaaac

SEQ ID NO: 13 - DNA, 20 bp, synthetic construct
  misc_feature 1..20: note = forward primer for vimentin
  source 1..20: mol_type = other DNA
gagaactttgccgttgaagc

SEQ ID NO: 14 - DNA, 20 bp, synthetic construct
  misc_feature 1..20: note = reverse primer for vimentin
  source 1..20: mol_type = other DNA
gcttcctgtaggtggcaatc

SEQ ID NO: 15 - DNA, 20 bp, synthetic construct
  misc_feature 1..20: note = forward primer for Snail
  source 1..20: mol_type = other DNA
cctccctgtcagatgaggac

SEQ ID NO: 16 - DNA, 20 bp, synthetic construct
  misc_feature 1..20: note = reverse primer for Snail
  source 1..20: mol_type = other DNA
ccaggctgaggtattccttg

SEQ ID NO: 17 - DNA, 20 bp, synthetic construct
  misc_feature 1..20: note = forward primer for Slug
  source 1..20: mol_type = other DNA
ggggagaagcctttttcttg

SEQ ID NO: 18 - DNA, 20 bp, synthetic construct
  misc_feature 1..20: note = reverse primer for Slug
  source 1..20: mol_type = other DNA
tcctcatgtttgtgcaggag

SEQ ID NO: 19 - DNA, 19 bp, synthetic construct
  misc_feature 1..19: note = forward primer for Twist-1
  source 1..19: mol_type = other DNA
cgacgagctggactccaag

SEQ ID NO: 20 - DNA, 19 bp, synthetic construct
  misc_feature 1..19: note = reverse primer for Twist-1
  source 1..19: mol_type = other DNA
cctccatcctccagaccga

SEQ ID NO: 21 - DNA, 20 bp, synthetic construct
  misc_feature 1..20: note = forward primer for Twist-2
  source 1..20: mol_type = other DNA
cagagcgacgagatggacaa

SEQ ID NO: 22 - DNA, 19 bp, synthetic construct
  misc_feature 1..19: note = reverse primer for Twist-2
  source 1..19: mol_type = other DNA
cacacggagaaggcgtagc

SEQ ID NO: 23 - DNA, 2507 bp, Homo sapiens
  source 1..2507: mol_type = genomic DNA
cagcacaccccggcacctcctctgcggcagctgcgcctcgcaagcgcagtgccgcagcgcacgccggagtggctgtagctgcccggcgcggcgccgccctgcgcgggctgtgggctgcgggctgcgcccccgctgctggccagctctgcacggctgcgggctctgcggcgcccggtgctctgcaacgctgcggcgggcggcatgggataacgcggccatggtgcgccgagatcgcctccgcaggatgagggagtggtgggtccaggtggggctgctggccgtgcccctgcttgctgcgtacctgcacatcccaccccctcagctctcccctgcccttcactcatggaagtcttcaggcaagtttttcacttacaagggactgcgtatcttctaccaagactctgtgggtgtggttggaagtccagagatagttgtgcttttacacggttttccaacatccagctacgactggtacaagatttgggaaggtctgaccttgaggtttcatcgggtgattgcccttgatttcttaggctttggcttcagtgacaaaccgagaccacatcactattccatatttgagcaggccagcatcgtggaagcgcttttgcggcatctggggctccagaaccgcaggatcaaccttctttctcatgactatggagatattgttgctcaggagcttctctacaggtacaagcagaatcgatctggtcggcttaccataaagagtctctgtctgtcaaatggaggtatctttcctgagactcaccgtccactccttctccaaaagctactcaaagatggaggtgtgctgtcacccatcctcacacgactgatgaacttctttgtattctctcgaggtctcaccccagtctttgggccgtatactcggccctctgagagtgagctgtgggacatgtgggcagggatccgcaacaatgacgggaacttagtcattgacagtctcttacagtacatcaatcagaggaagaagttcagaaggcgctgggtgggagctcttgcctctgtaactatccccattcattttatctatgggccattggatcctgtaaatccctatccagagtttttggagctgtacaggaaaacgctgccgcggtccacagtgtcgattctggatgaccacattagccactatccacagctagaggatcccatgggcttcttgaatgcatatatgggcttcatcaactccttctgagctggaaagagtagcttccctgtattacctcccctactcccttatgtgttgtgtattccacttaggaagaaatgcccaaaagaggtcctggccatcaaacataattctctcacaaagtccactttactcaaattggtgaacagtgtataggaagaagccagcaggagctctgactaaggttgacataatagtccacctcccattactttgatatctgatcaaatgtatagacttggctttgttttttgtgctattaggaaattctgatgagcattactattcactgatgcagaaagacgttcttttgcataaaagacttttttttaacactttggacttctctgaaatatttagaagtgctaatttctggcccacccccaacaggaattctatagtaaggaggaggagaaggggggctccttccctctcctcgaatgacgttatgggcacatgccttttaaaagttctttaagcaacacagagctgagtcctctttgtcatacctttggatttagtgtttcatcagctgtttttagttataaacattttgttaaaatagatattggtttaaatgatacagtattttaggtatgatttaagactatgatttacctatacattatatatattttataaagatactaaaccagcatacccttactctgccagagtagtgaagctaattaaacacatttggtttctgaataaattgaactaaatccaaactatttcctaaaatcacaggacattaaggaccaatagcatctgtgccagagatgtactgttattagctgggaagaccaattctaacagcaaataacagtctgagactcctcatacctcagtggttagaagcatgtctctcttgagctacagtagaggggaagggattgttgtgtagtcaagtcaccatgctgaatgtacactgattcctttatgatgactgcttaactccccactgcctgtcccagagaggctttccaatgtagctcagtaattcctgttactttacagacaggaaagttccagaaactttaagaacaaactctgaaagacctatgagcaaatggtgctgaatactttttttttaaagccacatttcattgtcttagtcaaagcaggattattaagtgattatttaaaattcgtttttttaaattagcaacttcaagtataacaactttgaaactggaataagtgtttattttctattaataaaaatgaattgtgacaaaaaaaaaaaaaaaaaaaaaaaaaaaaaaaaaaaaaaaaaaaaaaaaaa

SEQ ID NO: 24 - DNA, 2455 bp, Homo sapiens
  source 1..2455: mol_type = genomic DNA
agcacatgctgggctcgggggcgatgggcttgtgcgcggacctggcgacgctctagccccgagccgcgtattcgtggccgggtcctccctgggaacagggtgaaggccgagaacctctggcctcaggaagcgcatgcgcaaccggttctccgaaacatggagtcctgtaggcaaggtcttacctgaatcaggatgagggagtggtgggtccaggtggggctgctggccgtgcccctgcttgctgcgtacctgcacatcccaccccctcagctctcccctgcccttcactcatggaagtcttcaggcaagtttttcacttacaagggactgcgtatcttctaccaagactctgtgggtgtggttggaagtccagagatagttgtgcttttacacggttttccaacatccagctacgactggtacaagatttgggaaggtctgaccttgaggtttcatcgggtgattgcccttgatttcttaggctttggcttcagtgacaaaccgagaccacatcactattccatatttgagcaggccagcatcgtggaagcgcttttgcggcatctggggctccagaaccgcaggatcaaccttctttctcatgactatggagatattgttgctcaggagcttctctacaggtacaagcagaatcgatctggtcggcttaccataaagagtctctgtctgtcaaatggaggtatctttcctgagactcaccgtccactccttctccaaaagctactcaaagatggaggtgtgctgtcacccatcctcacacgactgatgaacttctttgtattctctcgaggtctcaccccagtctttgggccgtatactcggccctctgagagtgagctgtgggacatgtgggcagggatccgcaacaatgacgggaacttagtcattgacagtctcttacagtacatcaatcagaggaagaagttcagaaggcgctgggtgggagctcttgcctctgtaactatccccattcattttatctatgggccattggatcctgtaaatccctatccagagtttttggagctgtacaggaaaacgctgccgcggtccacagtgtcgattctggatgaccacattagccactatccacagctagaggatcccatgggcttcttgaatgcatatatgggcttcatcaactccttctgagctggaaagagtagcttccctgtattacctcccctactcccttatgtgttgtgtattccacttaggaagaaatgcccaaaagaggtcctggccatcaaacataattctctcacaaagtccactttactcaaattggtgaacagtgtataggaagaagccagcaggagctctgactaaggttgacataatagtccacctcccattactttgatatctgatcaaatgtatagacttggctttgttttttgtgctattaggaaattctgatgagcattactattcactgatgcagaaagacgttcttttgcataaaagacttttttttaacactttggacttctctgaaatatttagaagtgctaatttctggcccacccccaacaggaattctatagtaaggaggaggagaaggggggctccttccctctcctcgaatgacgttatgggcacatgccttttaaaagttctttaagcaacacagagctgagtcctctttgtcatacctttggatttagtgtttcatcagctgtttttagttataaacattttgttaaaatagatattggtttaaatgatacagtattttaggtatgatttaagactatgatttacctatacattatatatattttataaagatactaaaccagcatacccttactctgccagagtagtgaagctaattaaacacatttggtttctgaataaattgaactaaatccaaactatttcctaaaatcacaggacattaaggaccaatagcatctgtgccagagatgtactgttattagctgggaagaccaattctaacagcaaataacagtctgagactcctcatacctcagtggttagaagcatgtctctcttgagctacagtagaggggaagggattgttgtgtagtcaagtcaccatgctgaatgtacactgattcctttatgatgactgcttaactccccactgcctgtcccagagaggctttccaatgtagctcagtaattcctgttactttacagacaggaaagttccagaaactttaagaacaaactctgaaagacctatgagcaaatggtgctgaatactttttttttaaagccacatttcattgtcttagtcaaagcaggattattaagtgattatttaaaattcgtttttttaaattagcaacttcaagtataacaactttgaaactggaataagtgtttattttctattaataaaaatgaattgtgacaaaaaaaaaaaaaaaaaaaaaaaaaaaaaaaaaaaaaaaaaaaaaaaaaa

SEQ ID NO: 25 - DNA, 2419 bp, Homo sapiens
  source 1..2419: mol_type = genomic DNA
cgctgctgtggccaggcgtctggcatgctgaccacgtcgcgctgctgtaaaggaaacctgccccgcgcagcggcggtggctggagcgggagaaaccggactttgtgcaactttggccatagtggccatcccatgaatctgtttactagcttggtggatgagggagtggtgggtccaggtggggctgctggccgtgcccctgcttgctgcgtacctgcacatcccaccccctcagctctcccctgcccttcactcatggaagtcttcaggcaagtttttcacttacaagggactgcgtatcttctaccaagactctgtgggtgtggttggaagtccagagatagttgtgcttttacacggttttccaacatccagctacgactggtacaagatttgggaaggtctgaccttgaggtttcatcgggtgattgcccttgatttcttaggctttggcttcagtgacaaaccgagaccacatcactattccatatttgagcaggccagcatcgtggaagcgcttttgcggcatctggggctccagaaccgcaggatcaaccttctttctcatgactatggagatattgttgctcaggagcttctctacaggtacaagcagaatcgatctggtcggcttaccataaagagtctctgtctgtcaaatggaggtatctttcctgagactcaccgtccactccttctccaaaagctactcaaagatggaggtgtgctgtcacccatcctcacacgactgatgaacttctttgtattctctcgaggtctcaccccagtctttgggccgtatactcggccctctgagagtgagctgtgggacatgtgggcagggatccgcaacaatgacgggaacttagtcattgacagtctcttacagtacatcaatcagaggaagaagttcagaaggcgctgggtgggagctcttgcctctgtaactatccccattcattttatctatgggccattggatcctgtaaatccctatccagagtttttggagctgtacaggaaaacgctgccgcggtccacagtgtcgattctggatgaccacattagccactatccacagctagaggatcccatgggcttcttgaatgcatatatgggcttcatcaactccttctgagctggaaagagtagcttccctgtattacctcccctactcccttatgtgttgtgtattccacttaggaagaaatgcccaaaagaggtcctggccatcaaacataattctctcacaaagtccactttactcaaattggtgaacagtgtataggaagaagccagcaggagctctgactaaggttgacataatagtccacctcccattactttgatatctgatcaaatgtatagacttggctttgttttttgtgctattaggaaattctgatgagcattactattcactgatgcagaaagacgttcttttgcataaaagacttttttttaacactttggacttctctgaaatatttagaagtgctaatttctggcccacccccaacaggaattctatagtaaggaggaggagaaggggggctccttccctctcctcgaatgacgttatgggcacatgccttttaaaagttctttaagcaacacagagctgagtcctctttgtcatacctttggatttagtgtttcatcagctgtttttagttataaacattttgttaaaatagatattggtttaaatgatacagtattttaggtatgatttaagactatgatttacctatacattatatatattttataaagatactaaaccagcatacccttactctgccagagtagtgaagctaattaaacacatttggtttctgaataaattgaactaaatccaaactatttcctaaaatcacaggacattaaggaccaatagcatctgtgccagagatgtactgttattagctgggaagaccaattctaacagcaaataacagtctgagactcctcatacctcagtggttagaagcatgtctctcttgagctacagtagaggggaagggattgttgtgtagtcaagtcaccatgctgaatgtacactgattcctttatgatgactgcttaactccccactgcctgtcccagagaggctttccaatgtagctcagtaattcctgttactttacagacaggaaagttccagaaactttaagaacaaactctgaaagacctatgagcaaatggtgctgaatactttttttttaaagccacatttcattgtcttagtcaaagcaggattattaagtgattatttaaaattcgtttttttaaattagcaacttcaagtataacaactttgaaactggaataagtgtttattttctattaataaaaatgaattgtgacaaaaaaaaaaaaaaaaaaaaaaaaaaaaaaaaaaaaaaaaaaaaaaaaaa

SEQ ID NO: 26 - protein, 335 aa, Homo sapiens
MVRRDRLRRMREWWVQVGLLAVPLLAAYLHIPPPQLSPALHSWKSSGKFFTYKGLRIFYQDSVGVVGSPEIVVLLHGFPTSSYDWYKIWEGLTLRFHRVIALDFLGFGFSDKPRPHHYSIFEQASIVEALLRHLGLQNRRINLLSHDYGDIVAQELLYRYKQNRSGRLTIKSLCLSNGGIFPETHRPLLLQKLLKDGGVLSPILTRLMNFFVFSRGLTPVFGPYTRPSESELWDMWAGIRNNDGNLVIDSLLQYINQRKKFRRRWVGALASVTIPIHFIYGPLDPVNPYPEFLELYRKTLPRSTVSILDDHISHYPQLEDPMGFLNAYMGFINSF

SEQ ID NO: 27 - protein, 326 aa, Homo sapiens
MREWWVQVGLLAVPLLAAYLHIPPPQLSPALHSWKSSGKFFTYKGLRIFYQDSVGVVGSPEIVVLLHGFPTSSYDWYKIWEGLTLRFHRVIALDFLGFGFSDKPRPHHYSIFEQASIVEALLRHLGLQNRRINLLSHDYGDIVAQELLYRYKQNRSGRLTIKSLCLSNGGIFPETHRPLLLQKLLKDGGVLSPILTRLMNFFVFSRGLTPVFGPYTRPSESELWDMWAGIRNNDGNLVIDSLLQYINQRKKFRRRWVGALASVTIPIHFIYGPLDPVNPYPEFLELYRKTLPRSTVSILDDHISHYPQLEDPMGFLNAYMGFINSF

SEQ ID NO: 28 - protein, 326 aa, Homo sapiens; identical to SEQ ID NO: 27